SEQUENCE LISTING (2 sequences)

SEQ ID NO: 1 - protein, 400 aa, Artificial Sequence
  Homo sapiens
MVGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 375 bp, Homo sapiens
  primer_bind (75)..(99)
  misc_feature (135)..(136): cg06983746
ttaagggaaatatttttgtttttttttttttttatttttttttttttgattttgttttttaaagaaatatttatttattttgtagttgaagttgagaagttttaagaagtttagagtaaatttttggaagttttcgtttataattttttgataatagtttttaaggttttagttgttgtcgttgtgttatttaaataggttagtaggttatgtttatttttttgggattttgtagagtaggttagtatgggggtatagttttgttagtgtgaattgtttttagttataggagaatggttagtggggttataattttatgaattataattgtatgtttttttatatattataattgtataattttaagaattttat

(19) United States
(12) Patent Application Publication
(10) Pub. No.: US 2017/0106048 A1
(43) Pub. Date: Apr. 20, 2017

(54) Combination of CD95/CD95L inhibition and Cancer Immunotherapy

(71) Applicant: APOGENIX AG, HEIDELBERG (DE)

(72) Inventors: Claudia KUNZ, Lustadt (DE); Harald FRICKE, Mannheim (DE); Thomas HÖGER, Laudenbach (DE); Juergen GAMER, Dossenheim (DE)

(21) Appl. No.: 15/390,272

(22) Filed: Dec. 23, 2016

Related U.S. Application Data
(63) Continuation of application No. PCT/EP2015/064762, filed on Jun. 29, 2015.

(30) Foreign Application Priority Data
Jun. 27, 2014 (EP) 14174757.6

(51) Int. Cl. A61K 38/17; C07K 16/30; G01N 33/574; A61K 39/395; C07K 16/28

(52) U.S. Cl. CPC A61K 38/177; A61K 39/39558; C07K 16/2818; G01N 33/57492; C07K 16/3069; C07K 16/3046; C07K 16/3053; C07K 16/3023; C07K 16/3015; C07K 2317/31; C07K 2317/76; G01N 2333/70596

(57) ABSTRACT

The present invention relates to the treatment of cancer using a combination of an inhibitor of the CD95/CD95L signaling system and an immunotherapeutic agent, e.g. a cancer vaccine or a checkpoint inhibitor. Another aspect of the invention is the prognosis of responsiveness of a cancer to the treatment with a combination of a CD95 inhibitor and an immunotherapeutic agent. Further disclosed are preparations and kits for use in these methods.

This application is a continuation of PCT/EP2015/064762, filed Jun. 29, 2015; which claims priority of European Application No. 14174757.6, filed Jun. 27, 2014. The contents of the above applications are incorporated herein by reference in their entirety.

### REFERENCE TO SEQUENCE LISTING, TABLE OR COMPUTER PROGRAM

The Sequence Listing is concurrently submitted herewith with the specification as an ASCII formatted text file via EFS-Web with a file name of Sequence_Listing.txt with a creation date of Dec. 16, 2016, and a size of 6.09 kilobytes. The Sequence Listing filed via EFS-Web is part of the specification and is hereby incorporated in its entirety by reference herein.

### DESCRIPTION

The present invention relates to the treatment of cancer using a combination of an inhibitor of the CD95/CD95L signaling system and an immunotherapeutic agent, e.g. a cancer vaccine or a checkpoint inhibitor. Another aspect of the invention is the prognosis of responsiveness of a cancer to the treatment with a combination of a CD95L inhibitor and an immunotherapeutic agent. Further disclosed are preparations and kits for use in these methods.

The immune system has the capacity to recognize and destroy neoplastic cells; nevertheless, despite the fact that neoplastic transformation is associated with the expression of immunogenic antigens, the immune system often fails to respond effectively to these antigens. When this happens, the neoplastic cells proliferate uncontrollably leading to the formation of malignant cancers with poor prognosis for the affected individuals. Thus, engaging the immune system is deemed to be an essential step for cancer therapy to succeed.

Several strategies of cancer immunotherapy are currently under investigation. In general, cancer immunotherapy exploits the fact that cancer cells often have subtly different molecules on their surface that can be detected by the immune system. These molecules, known as cancer or tumor antigens, are most commonly proteins but also include other molecules such as carbohydrates. Immunotherapy is used to provoke (stimulate) the immune system into attacking the tumor cells by using these cancer antigens as targets.

Cancer vaccines try to get the immune system to mount an attack against cancer cells in the body. Instead of preventing disease, they are meant to get the immune system to attack a disease that already exists. Some cancer treatment vaccines are made up of cancer cells, parts of cells, or pure antigens. A vaccine may contain a cancer antigen as a protein or an immunogenic fragment thereof, or as RNA or DNA encoding the protein or as a vector containing said DNA, which stimulates the patient's immune system to attack tumors expressing the same antigen. Sometimes a patient's own immune cells are removed and exposed to these substances in vitro to create the vaccine. Once the vaccine is ready, it's injected into the body to increase the immune response against cancer cells. Vaccines often additionally comprise other substances or cells called adjuvants that help boost the immune response (more strongly) even further.

Cancer vaccines cause the immune system to attack cells with one or more specific antigens. If the appropriate response is stimulated, T lymphocytes (T cells) attack antigens directly, and provide control of the immune response. B cells and T cells develop that are specific for one antigen type. When the immune system is exposed to a different antigen, different B cells and T cells are formed. As lymphocytes develop, they normally learn to recognize the body's own tissues (self) as different from tissues and particles not normally found in the body (non-self). Once B cells and T cells are formed, a few of those cells will multiply and provide “memory” for the immune system. This allows the immune system to respond faster and more efficiently the next time it is exposed to the same antigen.

Several lines of evidence suggest that T cells are the main effectors in the immunological response against cancer cells. Immune regulatory proteins like indoleamine 2,3-dioxygenase (IDO), Cytotoxic T lymphocyte antigen 4 (CTLA-4) and Programmed cell death 1 ligand 1 (PD-L1) play a vital role in the immune suppression and tolerance induction of anti-cancer immune responses. CTLA-4 is a key negative regulator of T-cell responses, which can restrict the antitumor immune response.

Another approach of anticancer immunotherapy is called immune checkpoint blockade. To protect the body against disease, but without attacking healthy cells, the immune system uses multiple “checkpoint” systems. Some checkpoints stimulate immune responses while others inhibit them. Cancer cells can evolve means to evade checkpoints. Accordingly, so-called checkpoint modulators (CPMs) have been developed, that can reverse that effect, helping the immune system better fight the cancer.

A ligand-receptor interaction that has been investigated as a target for cancer treatment is the interaction between the transmembrane programmed cell death 1 protein (PD-1; also known as CD279) and its ligand, PD-1 ligand 1 (PD-L1). PD-1 is a regulatory surface molecule delivering inhibitory signals important to maintain T-cell functional silence against their cognate antigens. In normal physiology PD-L1 on the surface of a cell binds to PD-1 on the surface of an immune cell, which inhibits the activity of the immune cell. It appears that upregulation of PD-L1 on the cancer cell surface may allow them to evade the host immune system by inhibiting T cells that might otherwise attack the tumor cell. Expression of PD-L1 on tumors correlates with poor clinical outcome for a number of cancers including pancreas, renal cell, ovarian, head and neck, and melanoma. An inverse correlation was observed between PD-L1 expression and intraepithelial CD8+ T-lymphocyte count, suggesting that PD-L1 on tumor cells may suppress anti-tumor CD8+ T cells. Therefore, inhibitors of the PD-1/PD-L1 system are suggested as checkpoint modulators for use in cancer immunotherapy. For example antibodies that bind to either PD-1 or PD-L1 and thereby blocking this interaction may allow the T-cells to attack the tumor.

A major drawback of present cancer immunotherapy is that tumors co-opt existing mechanisms that are normally required to limit excessive inflammation and promote tissue recovery during infection or wound healing, and the execution of this program sustains tumor growth and promotes immunological tolerance. In addition, despite effective strategies to elicit an immune response, effective tumor control depends in part on the ability of tumor-reactive T-cells to infiltrate tumors.

It was found, that the tumor endothelium establishes a substantial barrier that limits T cell infiltration. Thus, efficient cancer immunotherapy depends on developing strategies to dismantle the tumor endothelial barrier. Recently, it was found that CD95L (also known as Apo-1 or FasL), an established homeostatic mediator of T cell apoptosis is expressed on the tumor endothelium of humans and mice. CD95L is upregulated by the cooperative action of proangiogenic and immunosuppressive paracrine factors in the tumor microenvironment (Motz et al., Nature Medicine, 2014, 20, 607-615). The CD95 positive tumor endothelium is described to be an active immune regulator that can directly suppress T cell function. Angiogenic growth factors induce CD95L expression on the tumor endothelium, which uniquely promotes an immunosuppressive and tolerogenic environment through preferential killing of tumor-reactive CD8+ cells.

In the present invention, it was surprisingly found that effectiveness of cancer immunotherapy can be significantly improved, if it is combined with the inhibition of the Thus, a first aspect of the present invention is a combination of an inhibitor of the CD95/CD95L signaling system and an immunotherapeutic agent for use in the treatment of cancer.

According to the invention, the inhibitor of the CD95/CD95L signaling system and the immunotherapeutic agent can be administered consecutively or simultaneously. Further, it is possible to use the inhibitor of the CD95/CD95L signaling system and the immunotherapeutic agent as two separate active agents or as a combined active agent having both CD95/CD95L inhibitory and immunotherapeutic activity.

Preferred inhibitors of the CD95/CD95L signaling system for use according to the present invention are inhibitory anti-CD95L-antibodies and antigen-binding fragments thereof as well as soluble CD95 molecules or CD95L-binding portions thereof. Examples of suitable inhibitory anti-CD95L antibodies are disclosed in EP-A-0 842 948, WO 96/29350, WO 95/13293. Also suitable are chimeric or humanized antibodies obtained therefrom, cf. e.g. WO 98/10070.

Further preferred are soluble CD95 receptor molecules, e.g. a soluble CD95 receptor molecule without transmembrane domain as described in EP-A-0 595 659 and EP-A-0 965 637 or CD95 receptor peptides as described in WO 99/65935, which are herein incorporated by reference.

Further preferred inhibitors are multimeric CD95 fusion polypeptides comprising the CD95 extracellular domain or a fragment thereof and a multimerization domain, particularly a trimerization domain, e.g. bacteriophage T4 or RB69 foldon fusion polypeptides as described in WO 2008/025516, which is herein incorporated by reference.

The CD95 ligand inhibitor FLINT or DcR3 or a fragment, e.g. a soluble fragment thereof, for example the extracellular domain optionally fused to a heterologous polypeptide, particularly a Fc immunoglobulin molecule is described in WO 99/14330, WO 99/50413 or Wroblewski et al., Biochem. Pharmacol. 65, 657-667 (2003), which are incorporated herein by reference. FLINT and DcR3 are proteins which are capable of binding the CD95 ligand and LIGHT, another member of the TNF family.

In a further embodiment of the present invention, the inhibitor is a CD95 inhibitor which may be selected from
(a) an inhibitory anti-CD95 receptor-antibody or a fragment thereof; and
(b) an inhibitory CD95 ligand fragment.

Examples of suitable inhibitory anti-CD95-antibodies and inhibitory CD95L fragments are described in EP-A-0 842 948 and EP-A-0 862 919 which are herein incorporated by reference.

In a still further embodiment of the present invention the inhibitor is a nucleic acid effector molecule. The nucleic acid effector molecule may be selected from antisense molecules, RNAi molecules and ribozymes which are capable of inhibiting the expression of the CD95 and/or CD95L gene.

In a still further embodiment the inhibitor may be directed against the intracellular CD95 signal transduction. Examples of such inhibitors are described in WO 95/27735 e.g. an inhibitor of the interleukin 1[beta] converting enzyme (ICE), particularly 3,4-dichloroisocoumarin, YVAD-CHO, an ICE-specific tetrapeptide, CrmA or usurpin (WO 00/03023). Further, nucleic acid effector molecules directed against ICE may be used.

In still a further embodiment, the inhibitor may be directed against a metalloproteinase (MMP), particularly against MMP-2 and/or MMP-9.

According to an especially preferred embodiment of the invention, the inhibitor of the CD95/CD95L signaling system is a CD95L inhibitor which comprises at least one extracellular domain of the CD95 molecule (particularly amino acids 1 to 172 (MLG . . . SRS) of the mature CD95 sequence according to U.S. Pat. No. 5,891,434) optionally fused to a heterologous polypeptide domain, particularly a Fc immunoglobulin molecule including the hinge region e.g. from the human IgG1 molecule. Particularly preferred fusion proteins comprising an extracellular CD95 domain and a human Fc domain are described in WO 95/27735, WO 2004/085478 and WO 2014/013039, which are incorporated herein by reference.

The CD95L inhibitor employed in the present invention can comprise a fusion protein comprising at least one extracellular CD95 domain or a functional fragment thereof and at least one Fc domain or a functional fragment thereof. In a particularly preferred embodiment, the CD95L inhibitor is or comprises a fusion protein selected from APG101, polypeptides having at least 70% identity to APG101 and functional fragments of APG101.

Fusion proteins comprising the extracellular domain of the death receptor CD95 (also called Apo-1 or Fas) fused to an immunoglobulin Fc domain are described in PCT/EP04/003239, the disclosure of which is included herein by reference. “Fusion protein”, as used herein, includes a mixture of fusion protein isoforms, each fusion protein comprising at least an extracellular CD95 domain (Apo-1; Fas) or a functional fragment thereof and at least a second domain being an Fc domain or a functional fragment thereof distributing within a pl range of about 4.0 to about 8.5. Accordingly, the extracellular CD95 domain as used herein may be also called “first domain”, while the Fc domain may be called “second domain”. Mixtures of CD95-Fc isoforms are particularly described in WO 2014/013039, the disclosure of which is incorporated herein by reference.

The first domain protein is an extracellular CD95 domain, preferably a mammalian extracellular domain, in particular a human protein, i.e. a human extracellular CD95 domain. The first domain, i.e. the extracellular CD95 domain, of the fusion protein preferably comprises the amino acid sequence up to amino acid 170, 171, 172 or 173 of human CD95 (SEQ ID NO. 1). A signal peptide (e.g. position 1-25 of SEQ ID NO: 1) may be present or not. Particularly for therapeutic purposes the use of a human protein is preferred.

The fusion protein can comprise one or more first domains which may be the same or different. One first domain, i.e. one extracellular CD95 domain, is preferred to be present in the fusion protein.

According to a preferred embodiment, the Fc domain or functional fragment thereof, i.e. the second domain of the fusion protein according to the invention, comprises the CH2 and/or CH3 domain, and optionally at least a part of the hinge region, or a modified immunoglobulin domain derived therefrom. The immunoglobulin domain may be an IgA, IgG, IgM, IgD, or IgE immunoglobulin domain or a modified immunoglobulin domain derived therefrom. Preferably, the second domain comprises at least a portion of a constant IgG immunoglobulin domain. The IgG immunoglobulin domain may be selected from IgG1, IgG2, IgG3 or IgG4 domains or from modified domains therefrom. Preferably, the second domain is a human Fc domain, such as a IgG Fc domain, e.g. a human IgG1 Fc domain.

The fusion protein can comprise one or more second domains which may be the same or different. One second domain, i.e. one Fc domain is preferred to be present in the fusion protein.

Further, both the first and second domains are preferably from the same species.

The first domain, i.e. the extracellular CD95 domain or the functional fragment thereof may be located at the N- or C-terminus. The second domain, i.e. the Fc domain or functional fragment may also be located at the C- or N-terminus of the fusion protein. However, the extracellular CD95 domain at the N-terminus of the fusion protein is preferred.

According to a further preferred embodiment, the fusion protein is APG101 (CD95-Fc, position 26-400 in SEQ ID NO: 1). As defined by SEQ ID NO: 1 APG101 can be a fusion protein comprising a human extracellular CD95 domain (amino acids 26-172) and a human IgG1 Fc domain (amino acids 172-400), further optionally comprising an N-terminal signal sequence (e.g. amino acids 1-25 of SEQ ID NO: 1). The presence of the signal peptide indicates the immature form of APG101. During maturation, the signal peptide is cleaved off. According to an especially preferred embodiment the signal sequence is cleaved off. APG101 with the signal sequence being cleaved off is also comprised by the term “unmodified APG101”.

In a further embodiment the fusion protein is a polypeptide having at least 70% identity, more preferably 75% identity, 80% identity, 85% identity, 90% identity, 95% identity, 96% identity, 97% identity, 98% identity, 99% identity with APG101. According to the present application the term “identity” relates to the extent to which two amino acid sequences being compared are invariant, in other words share the same amino acids in the same position.

The term “APG101” includes a fusion protein of position 26-400 of SEQ ID NO: 1, with and without a signal peptide. The term “APG101” also includes fusion proteins containing N-terminally truncated forms of the CD95 extracellular domain.

In another preferred embodiment the fusion protein according to the invention is a functional fragment of APG101. As used herein, the term “fragment” generally designates a “functional fragment”, i.e. a fragment or portion of a wild-type or full-length protein which has essentially the same biological activity and/or properties as the corresponding wild-type or full-length protein has.

A person skilled in the art is aware of methods to design and produce fusion proteins according to the present invention. The mixture of fusion protein isoforms, in particular APG101 isoforms, however, can be obtained by a method described, e.g., in PCT/EP04/03239, the disclosure of which is included herein by reference. According to a preferred embodiment designing a fusion protein for the use of the present invention comprises a selection of the terminal amino acid(s) of the first domain and of the second domain in order to create at least one amino acid overlap between both domains. The overlap between the first and the second domain or between the two first domains has a length of preferably 1, 2 or 3 amino acids. More preferably, the overlap has a length of one amino acid. Examples for overlapping amino acids are S, E, K, H, T, P, and D.

As indicated above, “fusion protein”, as used herein, includes a mixture of isoforms. The term “isoform” as used herein designates different forms of the same protein, such as different forms of APG101, in particular APG101 without signal sequence. Such isoforms can differ, for example, by protein length, by amino acid, i.e. substitution and/or deletion, and/or post-translational modification when compared to the corresponding unmodified protein, i.e. the protein which is translated and expressed from a given coding sequence without any modification. Different isoforms can be distinguished, for example, by electrophoresis, such as SDS-electrophoresis, and/or isoelectric focusing which is preferred according to the present invention.

Isoforms differing in protein length can be, for example, N-terminally and/or C-terminally extended and/or shortened when compared with the corresponding unmodified protein. For example, a mixture of APG101 isoforms according to the invention can comprise APG101 in unmodified form as well as N-terminally and/or C-terminally extended and/or shortened variants thereof. Thus, according to a preferred embodiment, the mixture according to the invention comprises N-terminally and/or C-terminally shortened variants of APG101. In particular preferred is a mixture of fusion protein isoforms comprising N-terminally shortened fusion proteins. Such N-terminally shortened fusion proteins may comprise −1, −2, −3, −4, −5, −6, −7, −8, −9, −10, −11, −12, −13, −14, −15, −16, −17, −18, −19, −20, −21, −22, −23, −24, −25, −26, −27, −28, −29, −30, −35, −40, −45 and/or −50 N-terminally shortened variants of unmodified APG101. Particularly preferred are −17, −21 and/or −26 N-terminally shortened variants. The numbering refers to the APG101 protein including signal sequence according to SEQ ID NO: 1. In other words, the shortened fusion proteins can comprise a sequence SEQ ID NO: 1 N-terminally truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 and/or 50 amino acids. Preferred shortened fusion proteins have SEQ ID NO: 1 N-terminally truncated by 16, 20, or 25 amino acids.

An example for a C-terminal shortening of APG101 isoforms is C-terminal Lys-clipping.

According to a preferred embodiment of the present invention the mixture of fusion proteins according to the present invention preferably comprises 50 mol-% unmodified APG101 in relation to modified isoforms, more preferably 40 mol-% unmodified APG101, more preferably 30 mol-% unmodified APG101, more preferably 20, more preferably 10 mol-% unmodified APG101, more preferably 5 mol-% unmodified APG101 and even more preferably 3 mol-% unmodified APG101 and most preferably 1 mol-% and/or less unmodified APG101. Most preferred is an embodiment comprising a mixture of fusion protein isoforms that does not comprise any unmodified APG101.

As outlined above, isoforms can also differ by amino acid substitution, amino acid deletion and/or addition of amino acids. Such a substitution and/or deletion may comprise one or more amino acids. However, the substitution of a single amino acid is preferred according to this embodiment.

Isoforms according to the invention can also differ with regard to post-translational modification. Post-translational modification according to the present invention may involve, without being limited thereto, the addition of hydrophobic groups, in particular for membrane localisation such as myristoylation, palmitoylation, isoprenylation or glypiation, the addition of cofactors for enhanced enzymatic activity such as lipoyation, the addition of smaller chemical groups such as acylation, formylation, alkylation, methylation, amidation at the C-terminus, amino acid addition, γ-carboxylation, glycosylation, hydroxylation, oxidation, glycilation, biotinylation and/or pegylation.

According to the present invention the addition of sialic acids, Fc-based glycosylation, in particular Fc-based N-terminal glycosylation, and/or pyro-Glu-modification are preferred embodiments of post-translational modifications.

According to a preferred embodiment the fusion proteins for use according to the present invention are comprised in a composition comprising high amounts of sialic acids. According to the present invention the content of sialic acid is preferably from about 4.0 to 7.0 mol NeuAc/mol APG101, more preferably from 4.5 to 6.0 mol NeuAc/mol APG101 and most preferably about 5.0 mol NeuAc/mol APG101. As used herein, sialic acids refer N- or O-substituted derivatives of neuraminic acid. A preferred sialic acid is N-acetylneuraminic acid (NeuAc). The amino group generally bears either an acetyl or glycolyl group but other modifications have been described. The hydroxyl substituents may vary considerably. Preferred hydroxyl substituents are acetyl, lactyl, methyl, sulfate and/or phosphate groups. The addition of sialic acid results generally in more anionic proteins. The resulting negative charge gives this modification the ability to change a protein's surface charge and binding ability. High amounts of sialic acid lead to better serum stability and thus, improved pharmacokinetics and lower immunogenicity. The high degree of sialylation of APG101 isoforms could be explained by the high amount of diantennary structure.

According to the present invention, glycosylation designates a reaction in which a carbohydrate is attached to a functional group of a fusion protein, functional fragment thereof as defined herein. In particular, it relates to the addition of a carbohydrate to APG101 or an isoform thereof. The carbohydrate may be added, for example, by N-linkage or O-linkage. N-linked carbohydrates are attached to a nitrogen of asparagine or arginine site chains. O-linked carbohydrates are attached to the hydroxy oxygen of serine, threonine, tyrosine, hydroxylysine or hydroxyproline side chains. According to the present invention, N-linkage, in particular Fc-based N-terminal glycosylation is preferred. Particularly preferred N-linked glycosylation sites are located at positions N118, N136 and/or N250 of APG101 (SEQ ID NO: 1).

Fucosylation according to the present invention relates to the adding of fucose sugar units to a molecule. With regard to the present invention such an addition of a fucose sugar unit to the fusion protein, and in particular to APG101, represents an especially preferred type of glycosylation. A high portion of fucosylated forms leads to a reduced antibody-dependent cellular cytotoxicity (ADCC). Thus, the mixture of fusion protein isoforms is characterised by reduced ADCC, which is beneficial for pharmaceutical and diagnostic applications.

Beside the first and second domain as defined herein, the fusion proteins for use according to the invention may comprise further domains such as further targeting domains, e.g. single chain antibodies or fragments thereof and/or signal domains. According to a further embodiment, the fusion protein used according to the invention may comprise an N-terminal signal sequence, which allows secretion from a host cell after recombinant expression. The signal sequence may be a signal sequence which is homologous to the first domain of the fusion protein. Alternatively, the signal sequence may also be a heterologous signal sequence. In a different embodiment the fusion protein is free from an additional N-terminal sequence, such as a signal peptide.

The fusion protein as described herein may be an N-terminally blocked fusion protein, which provides a higher stability with regard to N-terminal degradation by proteases, as well as a fusion protein having a free N-terminus, which provides a higher stability with regard to N-terminal degradation by proteases.

Modifications blocking the N-terminus of protein are known to a person skilled in the art. However, a preferred post-translational modification according to the present invention blocking the N-terminus is the pyro-Glu-modification. Pyro-Glu is also termed pyrrolidone carboxylic acid. Pyro-Glu-modification according to the present invention relates to the modification of an N-terminal glutamine by cyclisation of the glutamine via condensation of the α-amino group with a side chain carboxyl group. Modified proteins show an increased half-life. Such a modification can also occur at a glutamate residue. Particularly preferred is a pyro-Glu-modification, i.e. a pyrrolidone carboxylic acid, with regard to the N-terminally shortened fusion protein−26.

A mixture as described herein may comprise 80-99 mol-% N-terminally blocked fusion proteins and/or 1-20 mol-% fusion proteins having a free N-terminus.

According to a further preferred embodiment the mixture as described herein comprises 0.0 to 5.0 mol-%, more preferably 0.0 to 3.0 mol-% and even more preferably 0.0 to 1.0 mol-%, of fusion protein high molecular weight forms such as aggregates. In a preferred embodiment the mixture does not comprise any aggregates of fusion protein isoforms, in particular no dimers or aggregates of APG101. Dimers or aggregates are generally undesired because they have a negative effect on solubility.

The functional form of APG101 comprises two fusion proteins, as described herein, coupled by disulfide bridges at the hinge region at positions 179 or/and 182 with reference SEQ ID NO:1 of the two molecules. The disulfide bridge may also be formed at position 173 with reference to SEQ ID NO:1 of the two molecules, resulting in an improved stability. If the disulfide bridge at position 173 with reference to SEQ ID NO:1 is not required, the Cys residue at this position can be replaced by another amino acid, or can be deleted.

According to the invention, the mixture of fusion protein isoforms distributes within a pl range of about 4.0 to about 8.5. In a further embodiment the pl range of the mixture of fusion protein isoforms comprised by the composition according to the invention is about 4.5 to about 7.8, more preferably about 5.0 to about 7.5.

The isoelectric point (pi) is defined by the pH-value at which a particular molecule or surface carries no electrical charge. Depending on the pH range of the surrounding medium the amino acids of a protein may carry different positive or negative charges. The sum of all charges of a protein is zero at a specific pH range, its isoelectric point, i.e. the pl value. If a protein molecule in an electric field reaches a point of the medium having this pH value, its electrophorectic mobility diminishes and it remains at this site. A person skilled in the art is familiar with methods for determining the pl value of a given protein, such as isoelectric focussing. The technique is capable of extremely high resolution. Proteins differing by a single charge can be separated and/or fractionated.

According to the present invention, the inhibitor of the CD95/CD95L signaling system is combined with an immunotherapeutic agent. The immunotherapeutic agent for use according to the invention preferably comprises a cancer vaccine, and/or a checkpoint modulator. Also suitable are combinations of more than one cancer vaccine and/or checkpoint modulator.

A cancer vaccine for use according to the present invention may comprise one or more cancer antigens, in particular a protein or an immunogenic fragment thereof, DNA or RNA encoding said cancer antigen, in particular a protein or an immunogenic fragment thereof, cancer cell lysates, and/or protein preparations from tumor cells.

As used herein, a cancer antigen is an antigenic substance present in cancer cells. In principle, any protein produced in a cancer cell that has an abnormal structure due to mutation can act as a cancer antigen. In principle, cancer antigens can be products of mutated Oncogenes and tumor suppressor genes, products of other mutated genes, overexpressed or aberrantly expressed cellular proteins, cancer antigens produced by oncogenic viruses, oncofetal antigens, altered cell surface glycolipids and glycoproteins, or cell type-specific differentiation antigens.

Examples of cancer antigens include the abnormal products of ras and p53 genes. Other examples include tissue differentiation antigens, mutant protein antigens, oncogenic viral antigens, cancer-testis antigens and vascular or stromal specific antigens. Tissue differentiation antigens are those that are specific to a certain type of tissue. Mutant protein antigens are likely to be much more specific to cancer cells because normal cells shouldn't contain these proteins. Normal cells will display the normal protein antigen on their MHC molecules, whereas cancer cells will display the mutant version. Some viral proteins are implicated in forming cancer, and some viral antigens are also cancer antigens. Cancer-testis antigens are antigens expressed primarily in the germ cells of the testes, but also in fetal ovaries and the trophoblase. Some cancer cells aberrantly express these proteins and therefore present these antigens, allowing attack by T-cells specific to these antigens. Exemplary antigens of this type are CTAG1B and MAGEA1 as well as Rindopepimut, a 14-mer intradermal injectable peptide vaccine targeted against epidermal growth factor receptor (EGFR) vIII variant. Rindopepimut is particularly suitable for treating glioblastoma when used in combination with an inhibitor of the CD95/CD95L signaling system as described herein. Also, proteins that are normally produced in very low quantities, but whose production is dramatically increased in cancer cells, may trigger an immune response. An example of such a protein is the enzyme tyrosinase, which is required for melanin production. Normally tyrosinase is produced in minute quantities but its levels are very much elevated in melanoma cells. Oncofetal antigens are another important class of cancer antigens. Examples are alphafetoprotein (AFP) and carcinoembryonic antigen (CEA). These proteins are normally produced in the early stages of embryonic development and disappear by the time the immune system is fully developed. Thus self-tolerance does not develop against these antigens. Abnormal proteins are also produced by cells infected with oncoviruses, e.g. EBV and HPV. Cells infected by these viruses contain latent viral DNA which is transcribed and the resulting protein produces an immune response.

In addition to proteins, other substances like cell surface glycolipids and glycoproteins may also have an abnormal structure in tumor cells and could thus be targets of the immune system.

According to a preferred aspect of the invention, a cancer vaccine comprises a fusion protein of a portion of a cancer antigen and a heterologous fusion partner. It was found that such fusion proteins increase the immunogenicity of the cancer antigen and/or aid production of the protein in appropriate quantities and/or purity. See for example WO 99/40188 which describes a fusion protein of MAGE and, for example protein D a surface protein of the gram-negative bacterium, Haemophilus influenza B. The fusion protein can be prepared recombinantly and the protein D secretion sequence can be incorporated into the fusion protein to potentially assist secretion and solubilisation of the final product.

Checkpoint modulators for use according to the present invention preferably comprise antibodies directed against one or more checkpoint molecules, i.e. molecules involved in a “checkpoint” interaction of the immune system. These molecules serve as checks employed by the body to prevent a runaway immune response, which can be debilitating, and even deadly. Unfortunately, these necessary mechanisms of control can hinder the anti-cancer immune response. They can be harnessed by cancer cells as a defense against immune attack. Antibodies that bind checkpoint molecules and antagonize their activities can be designed to override these control mechanisms, disengaging the immune system's brakes or helping immune cells to overcome the molecular defenses of cancer cells.

FIG. 1 shows a diagram of several receptors involved in checkpoint interactions of the immune system. Preferred checkpoint modulators in terms of the present invention are agonists of the receptors CD28, Aux4, GITR, CD137, CD27 and/or HVEM. For example, agonistic antibodies binding to these receptors are suitable for use as checkpoint modulators. Alternatively, checkpoint modulators blocking the receptors CTLA-4, PD-1, TIM-3, BTLA, Vista and/or LAG3 or the interaction of these receptors with their respective ligands can be used. For example, antagonistic antibodies binding to these receptors or to their ligands are suitable for use as checkpoint modulators in terms of the invention.

A checkpoint modulator may for example comprise an inhibitor of the PD-1/PD-L1 receptor ligand interaction. Especially preferred is an antagonistic antibody specifically binding to PD-1 or PD-L1.

Further preferred checkpoint modulators for use according to the present invention are those comprising an inhibitor of CTLA-4. Blocking CTLA-4 was found to be a suitable means of inhibiting immune system tolerance to tumors and thereby providing a useful immunotherapy strategy for patients with cancer. Accordingly, a preferred checkpoint modulator is an antagonistic antibody specifically binding CTLA-4, e.g. ipilimumab.

Also preferred are checkpoint modulators inhibiting lymphocyte-activation gene 3 (LAG3), B7-H3, B7-H4 and/or T cell immunoglobulin mucin-3 (TIM3), e.g. antagonistic anti-CTLA-4 antibodies, anti-LAG3 antibodies, anti-B7-H3 antibodies, anti-B7-H4 antibodies and/or anti-TIM3 antibodies and combinations thereof. Another type of checkpoint modulators are co-stimulatory agents. T-cells require two signals to become fully activated. A first signal, which is antigen-specific, is provided through the T-cell receptor. A second signal, the co-stimulatory signal, is non-antigen-specific and is provided by the interaction between co-stimulatory molecules expressed on the membrane of APC and the T-cell. Exemplary co-stimulatory signals are provided by OX40L and CD40L. CD40 is a co-stimulatory protein found on antigen-presenting cells and its stimulation by CD40L is required for their activation. Particularly suitable for use as checkpoint modulators in the present invention are agonists, for example agonists of CD40.

According to another preferred embodiment of the invention, the combination of inhibitor of the CD95/CD95L signaling system and immunotherapeutic agent comprises a dual agent, i.e. a combined CD95/CD95L-inhibitor and immunotherapeutic agent. A dual agent may for example be a bispecific antibody, preferably a combined anti-CD95L and anti-checkpoint molecule antibody. Exemplary bispecific antibodies are those binding CD95L and a checkpoint molecule selected from PD-1, PD-L1, CTLA-4, LAG3, B7-H3, B7-H4 and/or TIM3.

According to the present invention, the inhibitor of the CD95/CD95L signaling system and the immunotherapeutic agent, i.e. cancer vaccine and/or checkpoint modulator, can be administered consecutively or simultaneously.

According to the present invention, the inhibitor of the CD95/CD95L signaling system and the immunotherapeutic agent, e.g. cancer vaccine and/or checkpoint modulator, can be used as a therapeutic composition including a combination of both types of active agents or as a kit for therapeutic use including both types of active agents separately, e.g. within at least two separate pharmaceutical compositions.

Thus, a further aspect of the present invention is a therapeutic composition or kit, comprising
(i) an inhibitor of the CD95/CD95L signaling system, and
(ii) an immunotherapeutic agent.

The inhibitor of the CD95/CD95L signaling system and the immunotherapeutic agent are as defined hereinabove.

According to a preferred embodiment of the invention, the CD95L inhibitor comprises a fusion protein comprising at least one extracellular CD95 domain or a functional fragment thereof and at least one Fc domain or a functional fragment thereof. In a particularly preferred embodiment, the CD95L inhibitor is or comprises a fusion protein selected from APG101, polypeptides having at least 70% identity to APG101 and functional fragments of APG101.

According to the invention, the inhibitor of the CD95/CD95L signaling system and the immunotherapeutic agent can be administered to a subject in need thereof, particularly a human patient, in a sufficient dose for the treatment of the specific conditions by suitable means. For example, the active agents for use according to the invention may be formulated as a pharmaceutical composition comprising the inhibitor of the CD95/CD95L signaling system and the immunotherapeutic agent together with pharmaceutically acceptable carriers, diluents and/or adjuvants. Alternatively, a kit comprising at least two separate pharmaceutical compositions can be provided, wherein one of the pharmaceutical compositions comprises the inhibitor of the CD95/CD95L signaling system and the other pharmaceutical composition comprises the immunotherapeutic agent, each together with pharmaceutically acceptable carriers, diluents and/or adjuvants.

Therapeutic efficiency and toxicity may be determined according to standard protocols. The inhibitor of the CD95/CD95L signaling system and/or the immunotherapeutic agent, e.g. a pharmaceutical composition comprising one or both active agents, may be administered systemically, e.g. intraperitoneally, intramuscularly, or intravenously or locally such as intranasally, subcutaneously or intrathecally. The dose of the active agent and/or composition administered will, of course, be dependent on the subject to be treated and on the condition of the subject such as the subject's weight, the subject's age and the type and severity of the disease or injury to be treated, the manner of administration and the judgement of the prescribing physician. For example, a daily dose of 0.001 to 100 mg/kg is suitable.

Of course, the use and/or pharmaceutical composition or kit according to the present invention may be combined with at least one further active agent. Which specific active agent is used depends on the indication to be treated. For example, cytotoxic agents such as doxorubicin, cisplatin or carboplatin, cytokines or other anti-neoplastic agents may be used in the treatment of cancer. Further, it is possible to use biologicals (e.g. antibodies or fusion proteins) such as but not limited to anti-angiogenic compounds (e.g. Avastin) or inhibitors of adhesion molecule, cytokine inhibitors or compounds addressing differentiation molecules (e.g. anti-CD20 [Rituximab] or anti-HER2 [Herceptin]).

It is understood, that the administration according to the present invention may be supported by other measurements for treating cancer, e.g. surgical interventions and/or radiation therapy.

The pharmaceutical composition or kit according to the invention may further comprise pharmaceutically acceptable carriers, diluents, and/or adjuvants. The term “carrier” when used herein includes carriers, excipients and/or stabilisers that are non-toxic to the cell or mammal being exposed thereto at the dosages and concentrations employed. Often, the physiologically acceptable carriers are aqueous pH buffered solutions or liposomes. Examples of physiologically acceptable carriers include buffers such as phosphate, citrate and other organic acids (however, with regard to the formulation of the present invention, a phosphate buffer is preferred); anti-oxidants including ascorbic acid, low molecular weight (less than about 10 residues) polypeptides; proteins such as serum albumin, gelatine or immunoglobulins; hydrophilic polymers such as polyvinyl pyrrolidone; amino acids such as glycine, glutamine, asparagine, arginine or lysine; monosaccharides, disaccharides, and other carbohydrates including glucose, mannose or dextrins, gelating agents such as EDTA, sugar, alcohols such as mannitol or sorbitol; salt-forming counterions such as sodium; and/or non-ionic surfactants such as TWEEN, polyethylene or polyethylene glycol.

A further aspect of the present invention is a method for the treatment of cancer, said method comprising
(a) administering an inhibitor of the CD95/CD95L signaling system, and
(b) administering an immunological agent.

The inhibitor of the CD95/CD95 signaling system and the immunological agent are as defined hereinabove.

According to a preferred embodiment of the invention, the CD95L inhibitor comprises a fusion protein comprising at least one extracellular CD95 domain or a functional fragment thereof and at least one Fc domain or a functional fragment thereof. In a particularly preferred embodiment, the CD95L inhibitor is or comprises a fusion protein selected from APG101, polypeptides having at least 70% identity to APG101 and functional fragments of APG101.

In the therapeutic uses and methods as described herein, the inhibitor of the CD95/CD95L signaling system and/or the immunotherapeutic agent is preferably administered at usual dosages that a person skilled in the art is aware of. The period of time in which the inhibitor of the CD95/CD95L signaling system and/or the immunotherapeutic agent is administered is preferably also the usual period of time for these compounds known to the person skilled in the art. As indicated above, not only the dosage of the administered composition used but also the dosage of the respective active agents, i.e. the inhibitor of the CD95/CD95L signaling system and/or the immunotherapeutic agent, may vary, depending, for example, on the specific active agents used, the method of administration and the judgment of a prescribing physician. The period of time in which each active agent is administered and the dosage of the active agent may vary, depending on the subject to be treated and on the condition of the subject, e.g. a subject's weight, the subject's age and the type and severity of the disease, in particular cancer, to be treated.

According to an especially preferred embodiment the inhibitor of the CD95/CD95L signaling system and the immunotherapeutic agent are administered simultaneously, e.g. as pharmaceutical composition comprising both active agents. Alternatively, the CD95/CD95L signaling system and the immunotherapeutic agent are administered immediately one after the other, e.g. using two separate pharmaceutical compositions.

According to another preferred embodiment of the invention, the inhibitor of the CD95/CD95L signaling system is administered first, i.e. before the administration of the immunotherapeutic agent.

According to another preferred embodiment of the invention, the immunotherapeutic agent is administered first, i.e. before the administration of the inhibitor of the CD95/CD95L signaling system.

The term “administered first” as used in the present application may describe an embodiment, wherein the inhibitor of the CD95/CD95L signaling system (or the immunotherapeutic agent) is administered at a dosage over a period of time which is considered to be a sufficient period of treatment to achieve a determinable effect. In case of the immunotherapeutic agent being administered first, the determinable effect may, for example, be tumor-specific antibodies/immune cells that can be detected. The inhibitor of the CD95/CD95L signaling system, e.g. the CD95L inhibitor, can then be administered to facilitate entry into the tumor. However, according to another embodiment of the present invention the first stage of treatment, in which the inhibitor of the CD95/CD95L signaling system (or the immunotherapeutic agent) is administered, may be terminated without the occurrence of a determinable effect. In this embodiment a determinable effect on the cancer or tumor cells to be treated will only occur after the application of the second compound, i.e. the immunotherapeutic agent. If treatment with the inhibitor of the CD95/CD95L signaling system (or with the immunotherapeutic agent) is finished, the immunotherapeutic agent (or the inhibitor of the CD95/CD95L signaling system, respectively) will be administered. The duration and dosage of the immunotherapeutic agent to be administered may correspond to the usual duration and dosage of an immunotherapeutic agent known to the person skilled in the art.

According to another embodiment, the cycle of administration of inhibitor of the CD95/CD95L signaling system and/or immunotherapeutic agent can be repeated at least once, if necessary, after a first cycle of administration of inhibitor of the CD95/CD95L signaling system and/or immunotherapeutic agent was completed.

The combination of at least one inhibitor of the CD95/CD95L signaling system and at least one immunotherapeutic agent, i.e. cancer vaccine and/or checkpoint modulator, was found to be suitable in the treatment of any type of cancer, in particular solid tumor tissue. The cancer to be treated may also be a cancer of lymphoid or myeloid origin. According to the present invention, the cancer is preferably selected from the group consisting of brain cancer, colon cancer, colorectal cancer, pancreatic cancer, breast cancer, lung cancer, renal cancer, liver cancer or/and metastatic disease thereof. More particular, the cancer disease is glioma, most particular glioblastoma.

It was found in the present invention that the combination of at least one inhibitor of the CD95/CD95L signaling system and at least one immunotherapeutic agent, i.e. cancer vaccine and/or checkpoint modulator, is particularly suitable for the treatment of CD95L positive cancer diseases as described herein below.

According to another preferred embodiment of the invention, the combination of at least one inhibitor of the CD95/C95L signaling system and at least one immunotherapeutic agent, i.e. cancer vaccine or checkpoint modulator, is particularly suitable for the treatment of cancer, wherein the methylation level of a preselected DNA sequence, in particular of a specific CpG site located upstream of and/or in a gene involved in CD95/CD95L signaling is 98%.

DNA methylation is a biochemical process which involves the addition of methyl groups to adenine or cytosine in the DNA. DNA methylation has been shown to play an important role, for example in developmental process and in regulation of gene expression. In this regard, methylation of cytocines of CpG sites within so called CpG islands is especially interesting. The term “CpG island” which is known to the person skilled in the art, denotes DNA regions which exhibit a higher frequency of the dinucleotide sequence CpG (a CpG site) compared to the corresponding frequency over the whole genome. In general, CpG islands are several hundred base pairs long and mostly found in the 5′ region of genes.

In the present invention, it was found that treatment with a combination of at least one inhibitor of the CD95/CD95L signaling system and at least one immunotherapeutic agent is particularly suitable for cancer diseases associated with a methylation level at defined CpGs of ≦98%, ≦95%, ≦90%, ≦85%, ≦80% or ≦75%. In this context, a methylation level of 100% denotes that in a given sample in all DNA copies the respective CpG sites are methylated.

The methylation level of a DNA sequence may be determined by any method known in the art. For example, the methylation level can be determined by the MassARRAY technique (Sequenom, San Diego, Calif., USA). This technique is based on detection of mass shifts introduced through sequence changes following bisulfite treatment.

The “DNA sequence located upstream of and/or in a gene involved in CD95/CD95L signaling” may be any type of DNA sequence. In this respect, “upstream of a gene” refers to the 5′ region of a gene. According to the present invention this DNA sequence may be part of a regulatory sequence and/or a CpG island, or it may comprise a regulatory sequence and/or a CpG island, as well as flanking regions. For example, the DNA sequence may comprise or be comprised by a regulatory sequence or the DNA sequence may comprise or be comprised by a CpG island. The length of the DNA sequence may depend on the specific type of cancer disease and/or the specific gene involved in CD95/CD95L signaling. For example, the DNA sequence may be >100 nucleotides long, preferably >50 nucleotides or >10 nucleotides. The DNA sequence can also be from 1-10 nucleotides in length. In the most preferred embodiment the DNA sequence to be methylated consists of one nucleotide. In this embodiment, the DNA sequence is C at position 135 in SEQ ID NO: 2, denoted as CpG1, and/or C at position 180 of SEQ ID NO: 2, denoted as CpG2 (based on Human February 2009 (GRCh37/hg19) Assembly), ranging from chr1:172,628,000-172,628,120 (reference genome GrCh37).

Another aspect of the present invention is a method of predicting responsiveness of a cancer disease to the treatment with a combination of a CD95L inhibitor and an immunotherapeutic agent, the method comprising
(a) determining the expression of CD95L in a cancer sample,
(b) classifying the cancer disease according to the level of CD95L expression,
(c) optionally determining the expression of at least one target molecule of the immunotherapeutic agent in said cancer sample and classifying the cancer disease according to the expression level of said target molecule,
(d) determining if the type of cancer that has been classified can be treated with a combination of a CD95L inhibitor and an immunotherapeutic agent, and optionally carrying out the treatment.

In a preferred embodiment of the present invention the method of predicting responsiveness of a cancer disease may include a step of determining and/or selecting a method of treatment which is suitable for the type of cancer that has been diagnosed and/or carrying out the treatment.

In the sense of the present application, “predicting responsiveness” means giving a prognosis on the responsiveness of a cancer disease. The terms “predicting” and “prognosing” are used interchangeably.

The immunotherapeutic agent preferably comprises a cancer vaccine and/or a checkpoint modulator as defined hereinabove.

The sample employed in the method for predicting responsiveness as described herein can be an archived tumor tissue, for example a biopsy or surgery material embedded in paraffin, which has been obtained in an earlier stage of the disease.

In the present invention, expression of CD95L can be determined by any known suitable method. For example, a suitable method may be a histological, histochemical and/or immunohistochemical method. According to one embodiment, the CD95L mRNA can be determined. A preferred example of a suitable method is a histological, histochemical or/and immunohistochemical method.

Alternatively, the expression of CD95L in the cancer sample can be determined by contacting the sample with an agent specifically binding to CD95L. For example, CD95L inhibitors, as disclosed herein, can be used for determination of CD95L, as these inhibitors can specifically bind to CD95L. Antibodies specifically binding to CD95L can be used. Suitable antibodies can be prepared by known methods. Further, suitable agents specifically binding to CD95L may include an extracellular receptor domain of CD95, or a functional fragment thereof, for example in a fusion polypeptide further comprising an Fc domain, or a functional fragment thereof. An example of a suitable fusion polypeptide is APG101, as described herein. Suitable labeling and staining methods are known.

According to a preferred embodiment of the invention, the CD95L inhibitor comprises a fusion protein comprising at least one extracellular CD95 domain or a functional fragment thereof and at least one Fc domain or a functional fragment thereof. In a particularly preferred embodiment, the CD95L inhibitor is or comprises a fusion protein selected from APG101, polypeptides having at least 70% identity to APG101 and functional fragments of APG101.

The cancer disease can be classified by the level of CD95L expression into a CD95L positive cancer disease or a CD95L negative cancer disease. In particular the CD95L positive cancer disease is characterized by a cell expressing CD95L on the cell surface.

A cancer can be regarded as CD95L positive, if at least 1%, at least 2%, at least 5%, at least 10%, at least 20%, or at least 50% of the cells in a cancer sample express CD95L. The number of CD95L positive cells can be determined by counting the cells in a microscopic section, or the CD95L positive cells can be quantified with the help of staining experiments.

CD95L expression is considered to be absent (CD95L negative) if essentially no cells expressing CD95L can be detected in the tissue sample, or if the sample is a sample which does not fulfil the criteria defined herein for a CD95L positive sample (non-positive sample). In a CD95L negative sample, the number of tumor cells expressing CD95L can be below the threshold defined herein for CD95L positive samples, for example below 1%, below 2%, below 3%, below 4%, below 5%, or below 10% of tumor cells.

A cancer can also be regarded as CD95L positive, if CD95L can be detected on at least 1%, at least 2%, at least 5%, at least 10%, at least 20%, or at least 50% of the area of tumor tissue in a tissue section. This value is termed herein as “% CD95L positive area of tumor tissue”. Non-tumor tissue is excluded in this analysis. A tissue section can be prepared by known methods. Suitable methods for detection of CD95L are described in PCT/EP2014/058746. CD95L expression can be considered to be absent (CD95L negative) if essentially no CD95L can be detected in the tissue sample, or if the value of % CD95L positive area of tumor tissue is below the threshold defined for a CD95 positive sample, for example below 1%, below 2%, below 3%, below 4%, below 5%, or below 10% of tumor area.

CD95L expression (e.g. in terms of cell number or surface in a tissue section) can be determined by known methods, for example by methods based upon automatized analysis of tissue sections.

By the method of the present invention, a prognosis of the responsiveness of any type of cancer, in particular solid tumor tissue, to the treatment with a CD95L inhibitor in combination with an immunotherapeutic agent can be provided. The cancer may also be a cancer of lymphoid or myeloid origin. Any type of cancer, in particular solid tumor tissue, can be determined to be CD95L expression positive or CD95L expression negative. The cancer can be characterized by invasive growth. The cancer disease for which a prognosis of the responsiveness is to be provided according to the present invention can be selected from the group consisting of brain cancer, colon cancer, colorectal cancer, pancreatic cancer, breast cancer, lung cancer, renal cancer, liver cancer or/and metastatic disease thereof. In particular, the cancer disease is glioma, more particular glioblastoma.

The cancer patient to be diagnosed or/and treated as described herein can be a patient with first or second relapse or progression of cancer, for example of glioblastoma. The patient may be a patient wherein standard treatment including radiotherapy (e.g. 60Gy) or/and temozolomide has failed, for example in the treatment of glioblastoma. In particular the patient is a candidate for re-irradiation, for example for treatment of glioblastoma.

Another aspect of the present invention is a method of predicting responsiveness of a cancer disease to the treatment with a combination of a CD95L inhibitor and an immunotherapeutic agent, the method comprising
(a) determining the methylation level of a DNA sequence located upstream of and/or in a gene involved in CD95/CD95L signaling in a sample obtained from a patient,
(b) classifying the cancer disease according to said methylation level,
(c) optionally determining the expression of a target molecule of the immunotherapeutic agent in said cancer sample and classifying the disease according to the expression level of said target molecule, and
(d) determing if the type of cancer that has been classified can be treated with a combination of a CD95L inhibitor and an immunotherapeutic agent and, optionally, carrying out the treatment.

In a preferred embodiment of the present invention the method of predicting responsiveness of a cancer disease may include a step of determining and/or selecting a method of treatment which is suitable for the type of cancer that has been diagnosed and/or carrying out the treatment.

The immunotherapeutic agent preferably comprises a cancer vaccine and/or a checkpoint modulator as defined hereinabove.

The sample employed in the method for predicting responsiveness as described herein can be an archived tumor tissue, for example a biopsy or surgery material embedded in paraffin, which has been obtained in an earlier stage of the disease.

The methylation level of a DNA sequence located upstream of and/or in a gene involved in CD95/CD95L signaling in a sample obtained from a patient can be done using any known suitable method.

According to a preferred aspect of the invention, the cancer is determined as responsive to treatment with a combination of a CD95L inhibitor and an immunotherapeutic agent if the methylation level is ≦98%, ≦95%, ≦90%, ≦85%, ≦80% or ≦75%. The “DNA sequence located upstream of and/or in a gene involved in CD95/CD95L signaling” is as described herein above. In the most preferred embodiment, the determination if cancer is responsive to treatment with a combination of a CD95L inhibitor and an immunotherapeutic agent is based on the methylation level of C at position 135 in SEQ ID NO:2, denoted CpG1 and/or C at position 180 of SEQ ID NO:2 denoted as CpG2 as described herein.

### BRIEF DESCRIPTION OF THE DRAWING

FIG. 1: Diagram showing several checkpoint interactions of the immune system. On the left side activating receptors are shown. Stimulating these receptors, for example using agonistic antibodies, is helpful for stimulating immune responses. On the right side inhibitory receptors are shown. Accordingly, blocking these receptors or interactions with these receptors is desirable. This can be done, for example, using blocking or antagonistic antibodies.

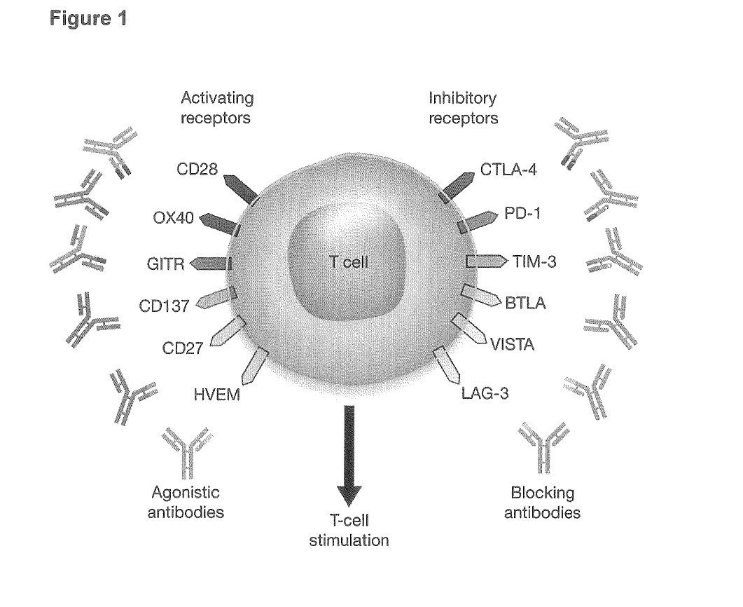

The invention is described in more detail by the following example.

### EXAMPLE

To evaluate the efficacy of the treatment of cancer diseases using a combination of an inhibitor of the CD95/CD95L signaling system and immunotherapeutic agent, we used a preclinical cancer model. The results obtained with a combination of the invention and the individual agents alone were compared. As the inhibitor of the CD95/CD95L signaling system we used APG101 and as the immunotherapeutic agent we used an inhibitor of PD-1 (programmed cell death protein 1).

Animals were treated with various tumor cell types to induce growth of ovarian cancer (ID-8 cells), colon cancer (CT-26 cells), melanoma (B-16 cells), breast cancer (4T1 cells) and lung cancer (Lewis lung cells). The mouse strains and cell types used to induce tumor formation are outlined in the following Tables 1-5. APG101 is applied alone in different doses or in combination with a PD-1 inhibitor compared to PD-1 inhibitor alone or vehicle alone according to the scheme presented in Tables 1-5.

Efficacy is followed by amount of tumor growth inhibiton, amount of infiltrating immune cells into the tumor and survival in the respective treatment groups. The results of the preclinical cancer models clearly show that the combination of an inhibitor of the CD95/CD95L signaling system and an immunotherapeutic agent is beneficial for the treated animals and better than using either agent alone. Benefit of such treatment was demonstrated by an increased infiltration of the tumor with immune cells, by a reduced growth of the tumor or by prolonged survival of the animals.

1. Combination of an inhibitor of the CD95/CD95L signaling system and an immunotherapeutic agent for use in the treatment of cancer.

2. The combination for the use of claim 1, wherein the inhibitor of the CD95/CD95L signaling system and the immunotherapeutic agent is administered consecutively or simultaneously, and wherein the combination comprises the use of the inhibitor of the CD95/CD95L signaling system and the immunotherapeutic agent as two separate active agents or as a combined active agent having both CD95/CD95L inhibitory and immunotherapeutic activity.

3. The combination for claim 1, wherein the inhibitor of the CD95/CD95L system comprises
(i) a fusion protein comprising at least one extracellular CD95 domain or a functional fragment thereof and at least one Fc domain or a functional fragment thereof and/or
(ii) an anti-CD95L specific antibody or a CD95L recognising fragment thereof.

4. The combination for the use of claim 3, wherein the fusion protein is selected from APG101, polypeptides having at least 70% identity to APG101 and functional fragments of APG101.

5. The combination for the use of claim 1, wherein the immunotherapeutic agent comprises a cancer vaccine and/or a checkpoint modulator.

6. The combination for the use of claim 5, wherein the cancer vaccine comprises at least one cancer antigen, in particular a protein or an immunogenic fragment thereof, DNA or RNA encoding said cancer antigen, in particular a protein or an immunogenic thereof, cancer cell lysates, and/or protein preparations from tumor cells.

7. The combination for the use of claim 1, wherein the immunotherapeutic agent comprises a checkpoint modulator selected from inhibitors of the interaction between PD-1 and PD-L1, e.g. antagonistic anti-PD-1 or anti-PD-L1 antibodies, inhibitors of CTLA-4, LAG3, B7-H3, B7-H4 and/or TIM3, e.g. antagonistic anti-CTLA-4 antibodies, anti-LAG3 antibodies, anti-B7-H3 antibodies, anti-B7-H4 antibodies and/or anti-TIM3 antibodies and combinations thereof.

8. The combination for the use of claim 1, wherein the combination comprises a bispecific antibody, preferably a combined anti-CD95L and checkpoint modulator antibody.

9. The combination for the use of claim 1, wherein the inhibitor of the CD95/CD95L system and the immunotherapeutic agent are provided as a therapeutic composition or as a kit for therapeutic use.

10. The combination for the use of claim 1, wherein the cancer is selected from the group consisting of brain cancer, colon cancer, colorectal cancer, pancreatic cancer, breast cancer, lung cancer, renal cancer, liver cancer or/and metastatic disease thereof.

11. The combination for the use of claim 1, wherein the cancer to be treated is a CD95L positive cancer and/or a cancer exhibiting a methylation level of a DNA sequence located upstream of and/or in a gene involved in CD95/CD95L signaling of is ≦98%, ≦95%, ≦90%, ≦85%, ≦80% or ≦75%.

12. The combination for the use of claim 11, wherein the CD95L positive cancer is characterized in that at least 1%, at least 2%, at least 5%, at least 10%, at least 20% or at least 50% of the cells in a cancer sample express CD95L and/or wherein the CD95L positive cancer is characterized in that CD95L can be detected on at least 1%, at least 2%, at least 5%, at least 10%, at least 20% or at least 50% of the area of tumor tissue in a tissue section from a patient to be treated.

13. The combination for the use of claim 11, wherein the DNA sequence located upstream of and/or in a gene involved in CD95/CD95L signaling comprises or is comprised by a regulatory sequence.

14. The combination for the use of claim 11, wherein the DNA sequence located upstream of and/or in a gene involved in CD95/CD95L signaling comprises or is comprised by a CpG island.

15. The combination for the use of claim 11, wherein the gene involved in CD95/CD95L signaling is coding for a protein selected from the group consisting of CD95, CD95L, Yes, FADD, GSκ-3 β, JNK, ERK 1/2, AKT and NF κ B.

16. The combination for the use of claim 11, wherein the DNA sequence located upstream of and/or in a gene involved in CD95/CD95L signaling consists of the C in the CpG site CpG1 corresponding to position 135 in SEQ ID NO:2 and/or the C in CpG site CpG2 corresponding to position 180 in SEQ ID NO:2.

17. A pharmaceutical composition or kit comprising
(i) an inhibitor of the CD95/CD95L signaling system, and
(ii) an immunotherapeutic agent selected.

18. The pharmaceutical composition or kit of claim 17, wherein the inhibitor of the CD95/CD95L signaling system and/or the immunotherapeutic agent are as defined in any one of claims 3 to 8.

19. A method of predicting responsiveness of a cancer disease to the treatment with a combination of a CD95L inhibitor and an immunotherapeutic agent, the method comprising
(a) determining the expression of CD95L in a cancer sample,
(b) classifying the cancer disease according the level of CD95L expression,
(c) optionally determining the expression of a target molecule of the immunotherapeutic agent in said cancer sample and classifying the cancer disease according to the expression level of said target molecule,
(d) determining if the type of cancer that has been classified can be treated with a combination of a CD95L inhibitor and an immunotherapeutic agent, and optionally carrying out the treatment.

20. The method of claim 19, wherein the expression of CD95L in the cancer sample is determined by contacting the sample with a CD95L inhibitor as defined in claim 3, 4 or 8.
